(19) United States
(12) Patent Application Publication
(10) Pub. No.: US 2010/0272794 A1
(43) Pub. Date: Oct. 28, 2010

(54) PHARMACEUTICAL COMPOSITION OF MEMANTINE

(76) Inventor: Aleksandra Dumicic, Zagreb (HR)

Correspondence Address: KENYON & KENYON LLP, ONE BROADWAY, NEW YORK, NY, 10004, US

(21) Appl. No.: 12/517,022

(22) PCT Filed: Nov. 15, 2007
(86) PCT No.: PCT/GB07/04360

§ 371 Date: May 29, 2009

(30) Foreign Application Priority Data
Nov. 30, 2006 (GB) 0623897.6

(51) Int. Cl. A61K 9/24; A61K 31/13; A61K 9/14; A61K 9/52; A61P 25/28

(52) U.S. Cl. 424/457; 514/662; 424/484; 424/472

(57) ABSTRACT

Present invention provides pharmaceutical composition comprising memantine or a pharmaceutically acceptable salt thereof as well as a method for its production. Also provided are different formulations of the composition their structure and preferred shape.

### FIELD OF INVENTION

The present invention relates to a pharmaceutical composition comprising memantine or a pharmaceutically acceptable salt thereof as an active ingredient and a process for production of the pharmaceutical composition. Preferred compositions use necessary pharmaceutically acceptable excipients that ensure adequate flowability of a tableting blend, required content uniformity in the composition and a desired drug release rate and stability of the final product.

### BACKGROUND OF THE INVENTION

Alzheimer's disease (AD) is an irreversible, progressive disorder in which brain cells (neurons) deteriorate, resulting in the loss of cognitive functions, primarily memory, judgment and reasoning, movement coordination, and pattern recognition. In advanced stages of the disease, all memory and mental functioning may be lost. A person with Alzheimer's disease has problems with memory, judgment, and thinking, which makes it hard for the person to work or take part in day-to-day life. The death of the nerve cells occurs gradually over a period of years. It is associated with senile dementia which is the mental deterioration (loss of intellectual ability) that is associated with old age. Two major types of senile dementia are identified: those due to generalized atrophy (Alzheimer type) and those due to vascular problems (mainly strokes). Senile dementia is often used when referring to Alzheimer's disease. Alzheimer's disease is most likely to affect older people: 20% of all people over 80 suffer from Alzheimer's disease. There is currently no cure for Alzheimer's disease, although there are drugs which offer symptomatic benefit.

Memantine is currently approved in Europe for the treatment of moderately severe to severe AD, and in the United States for the treatment of moderate to severe AD. In addition, memantine when given to moderate to severe AD patients receiving donepezil resulted in an unexpected greater relief of AD symptoms, compared to AD patients receiving placebo. This effect has not been demonstrated in patients with mild to moderate AD, where combination therapy involving administration of memantine and the other pharmaceutical compounds approved for treatment of AD did not result in any benefit compared with the used comparative compound.

WO 2005/06790B describes a method of treating mild-to-moderate Alzheimer's disease (AD) comprising administering to a subject in need thereof an effective amount of memantine or a pharmaceutically acceptable salt thereof. The method is directed to a group consisting of naive subjects and subjects who had previously been treated with other pharmaceutical compounds approved for treatment of AD but had discontinued AChEI therapy no later than one day prior to commencement of memantine administration.

WO 2006/009769 describes a pharmaceutically acceptable polymeric matrix carrier able to sustain memantine release rate from about 4 hours to about 24 hours following administration of the dosage form. However, it is known in the art that mechanisms of formation of the polymer matrices depend on numerous processing variables directly affecting drug release characteristics. This is particularly difficult in memantine compositions as the drug is soluble in water and highly permeable so any variation in matrix formation will most probably result in variation in both released and absorbed drug.

### DESCRIPTION OF THE INVENTION

Within this invention it has been found that replacing polymeric matrix with a lipidic core in memantine compositions minimizes undesired effects related to memantine water solubility. Therefore this invention is directed to providing a pharmaceutical composition comprising memantine or a pharmaceutically acceptable salt thereof, a lipidic drug release rate controlling substance and suitable pharmaceutical excipients.

Another direction of the present invention is to provide a pharmaceutical composition having extended release properties which provides a sustained release of memantine or a pharmaceutically acceptable salt thereof over an extended period of at least about 6 and up to about 30 hours, preferably up to about 28 hours and even more preferably up to about 24 hours. Such properties of the composition enable a patient to take the medicament only once daily instead of twice or three times a day as is currently the case. This would increase patient compliance and decrease the amount of negative consequences due to missed or mistimed dosages. A preferred composition contains a daily dosage of memantine or a pharmaceutically acceptable salt thereof, but does not release a substantial part of memantine, or a pharmaceutically acceptable salt thereof, rapidly and at once but releases it at a release rate over an extended period of time. The release rate should be such that at most 50% by weight of memantine or a pharmaceutically acceptable salt thereof present in the composition is released over the first 6 hours and preferably over the first 8 hours and even more preferably over the first 12 hours following entry of said composition into a use environment. Additionally the release rate should be such that at least 90% by weight and preferably 95% by weight and even more preferably 99% by weight of memantine or a pharmaceutically acceptable salt thereof present in the composition is released over about 16 and preferably over about 24 hours following entry of said composition into a use environment.

Memantine, or a pharmaceutically acceptable salt thereof, is highly soluble in aqueous media and polar solvents. The intended use environment for this composition is an aqueous environment. The high solubility of memantine makes it difficult to achieve a consistent drug release rate over extended periods. The drug release rate controlling substance protects memantine, or a pharmaceutically acceptable salt thereof, from direct and immediate contact with the aqueous solvent and therefore prevents the danger of a high dose dumping by immediate dissolution of a large part of the whole dosage of memantine or a pharmaceutically acceptable salt thereof from the composition. Lipidic drug release rate controlling substances according to the present invention are those which have the function of extending or prolonging the release of memantine, or a pharmaceutically acceptable salt thereof, so that the memantine, or a pharmaceutically acceptable salt thereof, is released over an extended period of time of at least 6 hours and up to 30 hours, preferably of at least 12 hours and up to 28 hours and even more preferably of at least 16 hours and up to 24 hours. The amount of lipidic drug release rate controlling substance varies according to the amount of memantine, or a pharmaceutically acceptable salt thereof, in the formulation, the other excipients and the type of drug release rate controlling substance used. Lipidic drug release rate controlling substances are preferably hydrophobic and function as core forming substances. Preferred lipids used as core forming drug release rate controlling substances, according to the present invention, can be selected from all pharmaceutically acceptable lipids with melting range from 35°-200° C. such as pharmaceutical fats, fatty acids, glycerides and waxes.

It is possible to provide a formulation with only one lipidic drug release rate controlling substance, the use of a combination of one or more different substances is possible. Optionally additional non-lipidic drug release rate controlling substances may be used in combination with the lipidic drug release rate controlling substances within the composition according to the present invention. Such combination offers even better drug release rate control and also greater possibilities for adjusting and managing drug release time intervals. Non-lipidic drug release rate controlling substances are not present inside or mixed with or in any other way involved in formation of the lipidic core. Preferred non-lipidic drug release rate controlling substances are polymers which function as film forming substances.

Preferred polymers used as film forming drug release rate controlling substances according to the present invention, are selected from the group consisting of cellulose based polymers (such as hydroxypropylmethylcellulose, hydroxypropylcellulose (HPC), hydroxyethylcellulose, methylcellulose, carboxymethylcellulose, ethylcellulose), polyethyleneglycol, polyvinylalcohol, chitosan, lactic acid, copolymers of lactic and glycolic acid, polymethacrylates, methacrylic acid copolymers, polyethylene glycol, xanthan gum, guar gum, and combinations thereof. Preferably it will be ethylcellulose.

To achieve the desired extended drug release rates and also different drug release rates the composition may be structured differently according to chosen drug release rate controlling substances.

Another direction of the present invention is to have said composition structured as a lipidic core system. It is preferably obtained by use of lipidic drug release rate controlling substance as a core forming substance. In such a composition, memantine, or a pharmaceutically acceptable salt thereof, is dispersed within the lipidic core. The dosage release properties of the core system may be dependent upon the solubility of the active ingredient in the lipidic core. Preferably the lipidic core is structured as a lipidic matrix. Incorporating lipidic matrices in memantine compositions minimizes effects related to memantine water solubility. Such a composition is typically formed by commonly used technologies such as wet granulation, melt granulation, dry granulation or direct compression of a lipid/drug mixture.

Another direction of the present invention is to have said composition structured as a combined “core-reservoir” system. In preferred case of lipidic core being structured as a lipidic matrix the system can also be referred to as “matrix-reservoir” system. The “core-reservoir” system is preferably obtained by use of both lipidic and additional non-lipidic drug release rate controlling substances. The lipidic drug release rate controlling substance forms a core, preferably structured as matrix, with active ingredient being dispersed in it, active ingredient being memantine or a pharmaceutically acceptable salt thereof according to the present invention. Non-lipidic drug release rate controlling substances form a film which surrounds the lipidic matrix entirely. Polymer coating further enables prolonged drug release rate.

The term “extended release properties” means that the memantine composition is neither formulated as immediate release nor as delayed release composition. Instead, it is formulated in order to have active ingredient released in a prolonged way so as to provide sustained release of memantine over an extended period of time. This term “extended” release is known in the art and may be interchangeably used with “prolonged”, “controlled” and “sustained” release.

The term “use environment” as used herein and above means human gastrointestinal tract. The preferred route of administration of the composition, according to the present invention, is through the gastrointestinal tract which can therefore be considered to be a use environment for the composition.

The term “environment” means media and conditions the same or substantially the same as conditions present in the human gastrointestinal tract or some of its parts.

The term “media” means different secretions present in the gastrointestinal tract such as gastric acid, different enzymes, intrinsic factor or mucus.

The term “conditions” means various physical and chemical properties of the above mentioned media such as polarity, acidity (pH), concentration or temperature.

The preferred use environment is the same or substantially the same as the environment of a human stomach comprising gastric juice. The gastric juice is aqueous solution of a strong gastric acid (0.1 M hydrochloric acid), digestive enzymes and mucus and has pH from 1 to 3. For the purposes of experiments and in vitro studies during the development of composition “use environment” preferably means water or 0.1 M hydrochloric acid as described in Test for disintegration of tablets and capsules (2.9.1) according to European Pharmacopoeia 5.05 which simulates gastric fluid present in human stomach or artificial gastric juice as described in European Pharmacopoeia 5.08 (1039900).

The term “pharmaceutical composition” as used herein and above means an oral dosage form comprised of a safe and effective amount of a memantine or a pharmaceutically acceptable salt thereof, drug release rate controlling substances and pharmaceutically acceptable excipients.

The term “safe and effective amount”, as used herein, means an amount of a compound or composition high enough to significantly positively modify the symptoms and/or condition to be treated, but low enough to avoid serious side effects (at a reasonable benefit/risk ratio), within the scope of sound medical judgment. The safe and effective amount of active ingredient for use in the method of the invention herein will vary with the particular condition being treated, the age and physical condition of the patient being treated, the severity of the condition, the duration of the treatment, the nature of concurrent therapy, the particular active ingredient being employed, the particular pharmaceutically-acceptable excipients utilized, and like factors within the knowledge and expertise of the attending physician.

The term “pharmaceutically acceptable excipients” as used herein and above includes any physiologically inert and pharmacologically inactive material known in the art, which is compatible with the physical and chemical characteristics of the particular memantine or a pharmaceutically acceptable salt thereof selected for use. Pharmaceutically acceptable excipients include, but are not limited to, polymers, resins, plasticizers, fillers, lubricants, disintegrants, binders, antiadherents, solvents, buffer systems, surfactants, preservatives or pharmaceutical grade dyes or pigments, and viscosity agents.

The term “about”, as used herein and above, means within 10%, preferably within 5%, and more preferably within 1% of a given value or range. Alternatively, the term “about” means within an acceptable standard error of the mean.

The pharmaceutical composition described herein comprises of from 0.5 to 40% by weight, preferably from 5 to 30% by weight; more preferably from 10 to 20% by weight of memantine or a pharmaceutically acceptable salt thereof. Further the pharmaceutical composition described herein comprises from 30 to 80% by weight, preferably from 35 to 70% by weight of one or more drug release rate controlling substances within which it preferably comprises from 30 to 70% by weight, more preferably from 40 to 65% by weight of lipidic drug release rate controlling substance and optionally from 10 to 30% by weight, preferably from 15 to 25% by weight of non-lipidic drug release rate controlling substance. Further the pharmaceutical composition described herein comprises from 20 to 50% by weight, preferably from 25 to 45% by weight and more preferably 30 to 40% by weight of one or more pharmaceutically acceptable excipients.

According to an advantageous composition of the present invention, it is to be administered twice a day, preferably once daily. Therefore a total amount of the memantine or a pharmaceutically acceptable salt thereof will depend on the prescribed daily dosage.

Filler can be one or more of the following; lactose monohydrate, lactose anhydrate, starch, sugar or sugar alcohols (such as glucose, sucrose, sorbitol, mannitol), celluloses (in powder forms of different types (eg. microcrystalline cellulose)), dicalcium phosphate dihydrate, hydroxypropyl cellulose, hydroxypropylmethyl cellulose or other cellulose ethers, vinylpyrrolidone containing polymers. Preferably it will be microcrystalline cellulose and lactose monohydrate. The total amount of filler present in the final composition is 15 to 55% by weight, preferably 20 to 40% by weight.

Binder can be one or more of the following; polyvidone, cellulose derivatives, polymethacrylates, starch and starch derivatives, gelatin, sucrose, acacia, tragacanth and sodium alginate. Preferably it will be starch in the form of pregelatinised starch. The total amount of binder present in the final composition is 1 to 30% by weight, preferably 1 to 20% by weight.

Glidant can be one or more of the following; stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc. Preferably it will be talc. The total amount of glidant present in the final composition is 1 to 10% by weight, preferably 1 to 5% by weight.

Lubricant can be one or more of the following; stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc. Preferably lubricant is magnesium stearate. The total amount of lubricant present in the final composition is 0.1 to 5% by weight preferably 0.5 to 3% by weight.

As is obvious from the above, the same excipient may have multiple functions. It is understood in the art that a function of a particular excipient in the composition can be dependant on the percentage of the excipient present in the composition or on possible interactions or interplays with other present excipients.

Another direction of the present invention is to provide the given composition in one of following forms; tablet or in form of tablets inside a capsule or in form of a powder inside a capsule. The preferred form is that of a tablet and more preferably in form of a film coated tablet. The tablet can be of round or oval biconvex shape with optionally scored or debossed sides if desired. The preferred shape of a tablet is round.

The formulation of the given composition may be obtained by commonly used technologies such as dry granulation, wet granulation and melt granulation. Preferably it is prepared by means of wet granulation; a process that includes:
a. intermixing of memantine or a pharmaceutically acceptable salt thereof with one or more fillers, glidants and lubricants and with one or more lipidic drug release rate controlling substances;
b. mixing and granulation by addition of water or aqueous solution of a suitable binder;
c. drying and milling of above obtained granules;
d. optional integration of additional lipidic drug release rate controlling substances and homogenization;
e. final blend is either compressed into tablets or filled in the capsules
f. where the composition is a tablet, optionally, applying additional non-lipidic drug release rate controlling substances in form of film or coating to the tablet.

### EXAMPLES

### Example 1

### Lipidic Matrix System

| Composition of the tablets | (mg) |
|---|---|
| Memantine | 20.00 |
| Microcrystalline cellulose | 20.00 |
| Lactose | 20.00 |
| Glyceryl tristearate | 30.00 |
| Hydrogenated vegetable oil NF, Type I | 30.00 |
| Talc | 3.00 |
| Magnesium stearate | 2.00 |

Intermix memantine with microcrystalline cellulose, lactose and glyceryl tristearate and granulate in a high-shear mixer with the addition of water in an amount sufficient to produce granules. Dry the wet granules in a fluid-bed dryer and mill through a 20 mesh (0.8 mm) screen.

Mix the granules with hydrogenated vegetable oil NF, Type I, talc and magnesium stearate and homogenize for 15 minutes. The final blend can be either compressed into tablets or filled in to capsules.

### Example 2

### Combined Lipidic Matrix-Reservoir System

| Composition of the tablets | (mg) |
|---|---|
| Memantine | 20.00 |
| Microcrystailine cellulose | 20.00 |
| Lactose | 20.00 |
| Glyceryl tristearate | 30.00 |
| Hydrogenated vegetable oil NF, Type I | 30.00 |
| Talc | 3.00 |
| Magnesium stearate | 2.00 |
| Ethylcellulose | 30.00 |

Intermix memantine with microcrystalline cellulose, lactose and glyceryl tristearate and granulate in a high-shear mixer with the addition of water in an amount sufficient to produce granules. Dry the wet granules in a fluid-bed dryer and mill through a 20 mesh (0.8 mm) screen.

Mix the granules with hydrogenated vegetable oil NF, Type I, talc and magnesium stearate and homogenize for 15 minutes. Coat the tablets using aqueous dispersion of ethylcellulose.

1. A pharmaceutical composition comprising memantine or a pharmaceutically acceptable salt thereof, a lipidic drug release rate controlling substance and suitable pharmaceutical excipients.

2. A composition as claimed in claim 1 having extended release properties.

3. A composition as claimed in claim 1 wherein memantine is released over an extended period of time of at least about 6 and up to about 30 hours.

4. A composition as claimed in claim 3 wherein memantine or a pharmaceutically acceptable salt thereof present in the composition is released over an extended period of time up to about 28 hours.

5. A composition as claimed in claim 1 wherein at most 50% by weight of memantine, or a pharmaceutically acceptable salt thereof, present in the composition is released over 8 hours following entry of said composition into a use environment.

6. A composition as claimed in claim 1 wherein at least 90% by weight of memantine or a pharmaceutically acceptable salt thereof present in the composition is released over about 24 hours following entry of said composition into a use environment.

7. A composition as claimed in claim 1 wherein at least 99% by weight of memantine or a pharmaceutically acceptable salt thereof present in the composition is being released over 24 hours following entry of said composition into a use environment.

8. A composition as claimed in claim 1 comprising from 0.5 to 30% by weight of memantine or a pharmaceutically acceptable salt thereof.

9. A composition as claimed in claim 1 wherein the lipidic drug release rate controlling substance is hydrophobic and can be selected from all pharmaceutically acceptable lipids with melting range from 35°-200° C. such as pharmaceutical fats, fatty acids, glycerides and waxes.

10. A composition as claimed in claim 1 structured as a lipidic core system.

11. A composition as claimed in claim 10 wherein the lipidic drug release rate controlling substance is used as a core forming substance.

12. A composition as claimed in claim 10 wherein the lipidic core system is structured as a matrix.

13. A composition as claimed in claim 1 wherein both lipidic and non-lipidic drug release rate controlling substances are used.

14. A composition as claimed in claim 13 wherein the non-lipidic drug release rate controlling substance is a polymer.

15. A composition as claimed in claim 14 wherein the polymer is selected from the group consisting of cellulose based polymer, polyethyleneglycol, polyvinylalcohol, chitosan, lactic acid, copolymers of lactic and glycolic acid, polymethacrylates, methacrylic acid copolymers, polyethylene glycol, xanthan gum, guar gum, and combination thereof.

16. A composition as claimed in claim 14 structured as a combined core-reservoir system.

17. A composition as claimed in claim 16 wherein the non-lipidic drug release rate controlling substance is a film forming substance.

18. A composition as claimed in claim 17 structured as a coated system.

19. A composition as claimed in claim 16 wherein memantine or a pharmaceutically acceptable salt thereof is being dispersed in the lipidic core.

20. A composition as claimed in claim 1 comprising from 30 to 80% by weight of one or more drug release rate controlling substances.

21. A composition as claimed in claim 20 comprising from 30 to 70% by weight of the lipidic drug release rate controlling substance.

22. A composition as claimed in claim 21 comprising from 40 to 65% by weight of the lipidic drug release rate controlling substance.

23. A composition as claimed in claim 20 optionally comprising from 10 to 30% by weight of a polymeric drug release rate controlling substance.

24. A composition as claimed in claim 23 optionally comprising from 15 to 25% by weight of the polymeric drug release rate controlling substance.

25. A composition as claimed in claim 1 wherein said suitable pharmaceutical excipients comprise one or more of the following: filler, binder, glidant and lubricant

26. A composition as claimed in claim 25 wherein the filler can be one or more of the following: lactose monohydrate, lactose anhydrate, starch, sugar and sugar alcohols (such as glucose, sucrose, sorbitol, mannitol), celluloses, dicalcium phosphate dihydrate, hydroxypropyl cellulose, hydroxypropylmethyl cellulose or other cellulose ethers, and vinylpyrrolidone containing polymers.

27. A composition as claimed in claim 26 comprising from 20 to 40% by weight of filler.

28. A composition as claimed in claim 25 wherein the binder can be one or more of the following: polyvidone, cellulose derivatives, polymethacrylates, starch and starch derivatives, gelatin, sucrose, acacia, tragacanth and sodium alginate.

29. A composition as claimed in claim 28 comprising from 1 to 20% by weight of binder.

30. A composition as claimed in claim 25 wherein the glidant can be one or more of the following: stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc

31. A composition as claimed in claim 30 comprising from 1 to 10% by weight of glidant.

32. A composition as claimed in claim 25 wherein the lubricant can be one or more of the following: stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc.

33. A composition as claimed in claim 30 comprising from 0.1 to 5% by weight of lubricant.

34. A composition as claimed in claim 1 obtained by use of wet granulation technology.

35. A composition as claimed in claim 1 in the form of a tablet or in the form of tablets inside a capsule or in the form of a powder inside a capsule.

36. A composition as claimed in claim 35 in the form of a tablet.

37. A composition as claimed in claim 36 in the form of a film coated tablet.

38. A process for preparation of the composition claimed in claim 1 comprising following steps:
a. intermixing memantine or a pharmaceutically acceptable salt thereof with one or more fillers, glidants and lubricants and with one or more lipidic drug release rate controlling substances;
b. mixing and granulation by addition of water or aqueous solution of a suitable binder;
c. drying and milling of above obtained granules;
d. optional integration of lipidic drug release rate controlling substances and homogenization;
e. final blend is either compressed into tablets or filled in to capsules;
f. where the composition is a tablet, optionally, applying non-lipidic drug release rate controlling substances in form of film or coating to the tablet.
